(19) United States
(12) Patent Application Publication
(10) Pub. No.: US 2014/0271697 A1
(43) Pub. Date: Sep. 18, 2014

(54) ENHANCED EXPRESSION OF PICORNAVIRUS PROTEINS

(71) Applicant: NOVAVAX, INC., Gaithersburg, MD (US)

(72) Inventor: Michael J. MASSARE, Mt. Airy, MD (US)

(73) Assignee: NOVAVAX, INC., Gaithersburg, MD (US)

(21) Appl. No.: 14/202,338

(22) Filed: Mar. 10, 2014

Related U.S. Application Data
(60) Provisional application No. 61/790,114, filed on Mar. 15, 2013.

(51) Int. Cl. C07K 14/005; A61K 47/48; A61K 39/135

(52) U.S. Cl. CPC C07K 14/005; A61K 39/135; A61K 47/4833
USPC 424/186.1; 530/350; 435/219; 536/23.4; 435/69.3

(57) ABSTRACT

The disclosure provides fusion proteins containing N-terminal signal peptides fused to immunogenic polypeptides. The immunogenic polypeptides may be from viruses, bacteria, or fungi. The disclosure also provides elevated expression of the immunogenic polypeptides using the N-terminal signal peptide. The N-terminal signal peptides enhance synthesis of the protein, particularly where the protein is neither a secretory nor a transmembrane peptide. The fusion proteins may be used to diagnose disease and to induce immune responses.

### CROSS REFERENCE TO RELATED APPLICATIONS

This application claims priority to U.S. Provisional Application Ser. No. 61/790,114, filed Mar. 15, 2013. The contents of which is hereby incorporated in full for all purposes.

### CROSS REFERENCE TO SEQUENCE LISTING

This application incorporates by reference the contents of the 24 kb Sequence Listing titled “NOVV_{—}52_SeqList.txt” created on Mar. 14, 2013.

### BACKGROUND

Humans and animals suffer from diseases and disorders caused by pathogens, such as bacteria, fungi, and viruses. The use of vaccines to induce immunity against infection by a pathogen is well known in the art. Vaccines have historically been prepared using a variety of approaches, including killed viruses and live attenuated vaccines. Killed viruses and live-attentuated viruses can be associated with deleterious effects, however, in some cases, the killed virus is not effective or indeed can exacerbate disease.

Efficiency of vaccine production can also be a barrier to commercial viability. In some cases, a long lead time as required before production of the vaccine, and in other vases preparing the live virus before inactivation can result in disease and disorder. For example, Foot-and-mouth disease virus (FMDV) is the prototypic aphthovirus within the family Picornaviridae. Economic losses from foot-and-mouth disease outbreaks are among the highest of all livestock diseases and widespread vaccination is the method of choice for disease control. The current vaccine is a killed whole virus vaccine with recognized limitations, such as those posed by growing the live virus prior to inactivation, poor growth of some strains, and reduced immunogenicity on storage. See Porta et al., “Efficient production of foot-and-mouth disease virus empty capsids in insect cells following down regulation of 3C protease activity,” J Virol Methods. 2013 February; 187(2):406-12.).

In recent years, the use of recombinant techniques to produce sub-unit vaccines has been particularly attractive. However, while recombinant approaches overcome issues with production of live virus and offer the potential for rapid synthesis, producing suitable amounts of immunogens can be challenging for a variety of reasons.

### SUMMARY OF THE INVENTION

The disclosure provides fusion proteins containing N-terminal signal peptides fused to polypeptides. The disclosure also provides methods of enhancing production of the polypeptide by using the N-terminal signal peptides, particularly where for proteins that are neither secretory nor transmembrane proteins. The fusion proteins may be used to diagnose disease and to induce immune responses. In some aspects, the polypeptides are immunogenic. The immunogenic polypeptides may be from viruses, bacteria, or fungi.

### BRIEF DESCRIPTION OF THE FIGURES

FIG. 1 illustrates the plasmid map of BV1168 (encoding SEQ ID NO:4) and BV 1169 (encoding SEQ ID NO:1) containing the FMDV P1 protein (shown as “vp1-4”), which is the uncleaved precursor polyprotein having, in order, vp0-vp3-vp1. vp0 contains vp4 and vp2. The sequences are identical except BV1169 contains the hemagglutinin signal peptide from A/Indonesia/5/05 influenza virus.

FIGS. 2A-2D illustrate the enhanced expression obtained when using an HA signal peptide compared to the same polypeptide expressed in the absence of a signal peptide. Plasmids were prepared as described in FIG. 1. BV1168 contained FMDV P1 protein with a hexa-histidine tag (His6). BV1169 contained FMDV P1 protein with a hexa-histidine tag (His6) and also the N-terminal signal peptide from A/Indonesia/5/05 HA (SEQ ID NO: 3). The plasmids were expressed in Sf9 cells and analyzed by SDS-PAGE and western blot. The lanes are as follows: M-Marker. Lanes 1-3 show FMDV P1 protein with a hexa-histidine tag, without the signal peptide, expressed from BV1168. Lanes 4-6 show FMDV P1 protein with a hexa-histidine tag and the signal peptide expressed from BV1169. Lane 7 shows a BV266 control and lane 8 shows BV 1064 S300 Fxn. FIG. 2A shows the enhanced expression in lanes 4-6 by total protein stain in SDS-PAGE. FIGS. 2B-D shows enhanced expression in lanes 4-6 detected by anti-Histidine antibody (FIG. 2B; “anti HIS Mab”), anti-FMDV vp2 antibody (FIG. 2C; “F1412SA Mab”), and by anti-FMDV vp1 antibody (FIG. 2D; “12FE9.2.1A Mab”).

FIG. 3 shows enhanced expression of individual non-secretory proteins by expressing them with a signal peptide. Sf9 cells were infected with Baculovirus expressing recombinant proteins with and without a signal peptide. Cells were harvested 70 hours post-infection. The harvested crude material (i.e., cells and medium) was analyzed by western blot using monoclonal antibody specific to the recombinant protein. Panel (a), the left panel, shows expression of FMDV vp0. Panel (b), the right panel, shows expression of FMDV vp1 protein “+” recombinant protein contained a signal peptide, “−” recombinant protein did not have a signal peptide.

FIGS. 4A-4D illustrates sequences disclosed herein. FIG. 4A shows the nucleotide sequence encoded by the BV1169 plasmid. The expressed protein is the FMDV P1 precursor polyprotein, which contains proteins vp1 to vp4 (i.e., vp0-vp3-vp1; vp0 contains both vp2 and vp4), along with an HA signal peptide on the N-terminus and a His6-tag on the C-terminus.

FIG. 4B shows the peptide sequence expressed by BV1169. The expressed protein is the FMDV P1 protein, which contains proteins vp1 to vp4, along with an HA signal peptide on the N-terminus and a His6-tag on the C-terminus. The signal peptide (MEKIVLLLAIVSLVK; SEQ ID NO:3) is from Indo H5N1 HA. FIGS. 4C and 4D shows the nucleotide and peptide sequence encoded by the BV1168 plasmid, respectively. The encoded sequence is the same as BV1169, except that BV 1168 lacks the HA signal peptide.

FIG. 5A-C illustrates expression of single proteins and proteins in tandem. Sf9 cells were infected with recombinant baculovirus (BV) expressing proteins 1, 2, 3, and/or 4 with (+) or without (−) a signal peptide and harvested ˜65 hrs post-infection. Crude samples (i.e. cells and medium) were harvested and analyzed by SDS-PAGE and western blots. The expressed recombinant proteins are indicated by the dot. BV1 expresses FMDV vp1. BV2 expresses FMDV vp0 and vp3. BV3 expresses FMDV vp1, vp0 and vp3. BV4 expresses the FMDV P1 polyprotein (i.e., vp0-vp3-vp1). FIG. 5A shows a total protein stain. FIGS. 5B and 5C show binding of vp1 and vp2 antibodies, respectively. Note that the vp0 protein contains the vp2 protein and the vp4 protein. The reaction of BV2 with the vp1 antibody is due to cross-reaction with vp0. Enhanced expression with the FMDV protease 3C was also obtained (not shown). “+” recombinant protein contained a signal peptide, “−” recombinant protein did not have a signal peptide.

### DETAILED DESCRIPTION OF THE INVENTION

### Definitions

As used herein, the term “baculovirus,” also known as baculoviridae, refers to a family of enveloped DNA viruses of arthropods, members of which may be used as expression vectors for producing recombinant proteins in insert cell cultures. The virion contains one or more rod-shaped nucleocapsids containing a molecule of circular supercoiled double-stranded DNA (Mw 54×10⁶-154×10⁶). The virus used as a vector is generally Autographa californica nuclear polyhedrosis virus (NVP). Expression of introduced genes is typically under the control of the strong promoter that normally regulates expression of the polyhedron protein component of the large nuclear inclusion in which the viruses are embedded in the infected cells.

As used herein, the term “derived from” refers to the origin or source of a protein, nucleic acid or other molecule. The proteins, nucleic acids, and other molecules may be altered from the source as described herein. In some aspects, the alteration includes deleting residues or nucleotides of a full-length sequence. In other aspects, the alterations include conservative mutations from the wild-type sequence.

As used herein, the term “vaccine” refers to a preparation containing an immunogen which is used to induce an immune response against the immunogen to provide a protective immune response (i.e., an immune response that reduces the severity of disease or disorder caused by a pathogen). The protective immune response may include formation of antibodies and/or a cell-mediated response. Preferably, the vaccine induces production of neutralizing antibodies. Depending on context, the term “vaccine” may also refer to a suspension or solution of an immunogen that is administered to a subject to produce protective immunity.

As used herein, the term “VLP” means virus-like particle.

As used herein, the term “about” means plus or minus 10% of the indicated value.

As used herein the term “adjuvant” refers to a compound that, when used in combination with an immunogen, modifies the immune response induced against the immunogen compared to an immune response against the immunogen administered without an adjuvant

As used herein an “effective dose” refers to an amount of an immunogen sufficient to induce an immune response that reduces at least one symptom of a disease or disorder induced by a bacterial toxin, such that the administered dose provides a therapeutic benefit in the treatment or management of a bacterial infection. An effective dose may be determined e.g., by measuring amounts of neutralizing secretory and/or serum antibodies, e.g., by plaque neutralization, complement fixation, enzyme-linked immunosorbent (ELISA), or microneutralization assay.

As used herein the term “substantially protective antibody response” refers to an immune response comprising the production of antibodies, for example, neutralizing antibodies, which blocks bacterial toxins from entering cells.

As used herein, the term “immunogen” or “antigen” refer to substances such as proteins, and peptides that are capable of eliciting an immune response. For example, in some aspects, the immunogen is an immunogenic polypeptide.

As used herein, the term “isolated” refers to a substance such as a nucleic acid, a protein, VLP, or the like that is not in a subject.

As use herein, the term “subject” or “patient” refers to any member of the subphylum cordata, including, without limitation, humans and other primates, including non-human primates such as chimpanzees and other apes and monkey species. In some aspects the suspect is a mammal. Suitable mammals include farm animals such as cattle, sheep, pigs, goats and horses and domestic mammals such as dogs and cats. In other aspects the subject is a laboratory animals rodents such as mice, rats and guinea pigs. Additional subjects include birds. The birds may be domestic, wild, or game birds. For example, chickens, turkeys and other gallinaceous birds, ducks, geese. The term encompasses adult and newborn individuals.

As used herein the term “avian influenza virus” refers to those influenza viruses found chiefly in birds but that can also infect humans or other animals. In some instances, avian influenza viruses may be transmitted or spread from one human to another. An avian influenza virus that infects humans has the potential to cause an influenza pandemic, i.e., morbidity and/or mortality in humans. A pandemic occurs when a new strain of influenza virus (a virus in which human have no natural immunity) emerges, spreading beyond individual localities, possibly around the globe, and infecting many humans at once.

As used herein the term “seasonal influenza virus” refers to the influenza viral strains that have been determined to be passing within the human population for a given influenza season based on epidemiological surveys conducted by National Influenza Centers worldwide. These meetings result in the selection of three viruses (two subtypes of influenza A viruses and one influenza B virus) to go into flu vaccines for the following fall and winter. The selection occurs in February for the northern hemisphere and in September for the southern hemisphere. Usually, one or two of the three virus strains in the vaccine changes each year. Influenza A viruses are divided into subtypes based on two proteins on the surface of the virus: the hemagglutinin (H) and the neuraminidase (N). There are 17 different hemagglutinin subtypes and 10 different neuraminidase subtypes. Influenza A viruses can be further broken down into different strains. Influenza B viruses are not divided into subtypes, but can be broken down into different strains. See “A revision of the system of nomenclature for influenza viruses: a WHO memorandum,” (1980) Bulletin of the World Health Organization, 58(4): 585-591.

As used herein the term “immunogenic polypeptide” refers to part or all of a protein from a pathogen that induces an immune response when administered to a subject. Suitable pathogens include viruses, fungi, and bacteria.

### Enhanced Expression of Proteins in Host Cells

Recombinant production of proteins in host cells can be hampered by low expression levels. The present disclosure provides methods of producing polypeptides more efficiently than current techniques by using signal peptides to increase expression. Signal peptides (sometimes referred to as signal sequence, leader sequence or leader peptide) are short peptides present at the N-terminus of the majority of newly synthesized proteins that are destined towards the secretory pathway. The core of a signal peptide contains a long stretch of hydrophobic amino acids that has a tendency to form a single alpha-helix. At the end of the signal peptide there is typically a stretch of amino acids that is recognized and cleaved by signal peptidase enzymes.

Increased expression of proteins is obtained by using the signal peptide compared to expressing the protein without the signal peptide. Polypeptides for expression are preferably derived from proteins that are non-secretory and those that are non-transmembrane.

In particular aspects, the present disclosure provides fusion proteins, and compositions containing fusion proteins, for inducing immune responses against pathogens. Also provided are nucleic acids encoding the fusion proteins, vectors containing the nucleic acids, and host cells containing the vectors. Using an N-terminal signal peptide provides elevated expression of a desired polypeptide such that sufficient production is achieved to provide commercially viable amounts of the polypeptide.

In particular aspects, the polypeptide is an immunogenic polypeptide derived from one or more pathogens that may be used to induce protective immune responses. The pathogen-derived polypeptides also have use as diagnostic reagents for example, in diagnosing FMDV infection. See Grubman and Baxt, “Foot-and-mouth disease,” Clin Microbiol Rev. 2004 April; 17(2):465-93.

Fusion Proteins with N-Terminal Signal Peptide

The polypeptide is typically expressed as a fusion protein. As used herein, the term “fusion protein” refers to a protein translated as a single polypeptide that contains polypeptide sequences from at least two different proteins connected via an amide bond. The fusion protein is typically prepared using classical molecular biology approaches used to prepare and express fusion proteins containing portions of at least two different proteins. In certain aspects, the fusion protein contains an N-terminal signal sequence and a C-terminal immunogenic polypeptide.

In some aspects, the signal peptide is derived from the HA protein of influenza strain A/Indonesia/5/05. For example, the signal peptide may consist of MEKIVLLLAIVSLVK (SEQ ID NO:3). In other aspects, the signal peptide comprises MEKIVLLLAIVSLVK. Additional amino acids C-terminal of the K residue in the HA protein (SEQ ID NO:6) may be used. In some aspects, the signal peptide contains an additional 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids of the HA protein.

HA sequence from A/Indonesia/5/05
SEQ ID NO: 6
MEKIVLLLAIVSLVKSDQICIGYHANNSTEQVDTIMEKNVTVTHAQDILE
KTHNGKLCDLDGVKPLILRDCSVAGWLLGNPMCDEFINVPEWSYIVEKAN
PTNDLCYPGSFNDYEELKHLLSRINHFEKIQIIPKSSWSDHEASSGVSSA
CPYLGSPSFFRNVVWLIKKNSTYPTIKKSYNNTNQEDLLVLWGIHHPNDA
AEQTRLYQNPTTYISIGTSTLNQRLVPKIATRSKVNGQSGRMEFFWTILK
PNDAINFESNGNFIAPEYAYKIVKKGDSAIMKSELEYGNCNTKCQTPMGA
INSSMPFHNIHPLTIGECPKYVKSNRLVLATGLRNSPQRESRRKKRGLFG
AIAGFIEGGWQGMVDGWYGYHHSNEQGSGYAADKESTQKAIDGVTNKVNS
IIDKMNTQFEAVGREFNNLERRIENLNKKMEDGFLDVWTYNAELLVLMEN
ERTLDFHDSNVKNLYDKVRLQLRDNAKELGNGCFEFYHKCDNECMESIRN

In some aspects, the immunogenic polypeptide contains two or more proteins aligned in tandem such that the several proteins form a continuous open reading frame. In certain aspects, the tandem proteins are referred to as a “polyprotein,” reflecting their production during normal infection. In some aspects, the proteins in the tandem polypeptide are purified from the host as a single polypeptide. In other aspects, the tandem polypeptide is cleaved during synthesis; for example, by endogenous proteases.

In some aspects, the fusion protein contains an immunogenic polypeptide derived from one pathogen. In other aspects, the immunogenic polypeptide may be a polyprotein comprising part or all of two, three, four, five or six pathogen proteins. In some aspects, the polyprotein may contain proteins derived from more than one pathogen. Thus, for example, the fusion protein may contain proteins derived from two pathogens, three pathogens, four pathogens, or five pathogens. The pathogens may be from the same species or from different species; for example, the fusion protein may contain protein derived from a virus, a bacteria, and a fungus. In particular aspects, the pathogens are different strains of the same pathogen species; for example, a fusion protein may contain portions of the same proteins from different HIV strains, thus providing for immunity against multiple strains when administered as a vaccine.

The immunogenic polypeptide may contain a fragment of a full-length protein. Variable sizes of fragments of the full-length protein are acceptable. The fragment may have at least 10 amino acids, at least 25 amino acids, at least 50 amino acids, at least 75 amino acids, at least 100 amino acids, at least 125 amino acids, at least 150 amino acids, at least 200 amino acids, at least 250 amino acids, at least 275 amino acids, at least 300 amino acids, at least 350 amino acids, at least 375 amino acids, or at least 400 amino acids of the full-length protein.

In other aspects, the immunogenic polypeptide shares identity with a native polypeptide. Identity may be measured using ClustalW (Version 2; ch.embnet.org/software/ClustalW.html) using the following parameters: Scoring matrix: BLOSUM; Opening gap penalty: 10; Extending gap penalty: 10; End gap penalty: 0.05; Separation gap penalty: 0.05. Compared to the native protein sequence, an immunogenic polypeptide may share at least 70% identity, at least 80% identity, at least 85% identity, at least 90% identity, at least 95% identity, at least 97% identity, at least 98% identity, at least 99% identity, where identity is measured over the length of the polypeptide.

In particular aspects, the immunogenic polypeptide is from a virus. Suitable viruses include picornaviridae (such as polio, HFMD, Coxsackievirus, enterovirus), Flaviviruses (such as hepatitis C, West Nile, and Dengue fever) and Togaviruses (such as alphaviruses and rubella). Proteins from human papilloma virus (HPV) may also be expressed at greater level using the fusion protein approach disclosed herein.

In particular aspects, the virus is a picornavirus. Picornavirus replication initiates from the translation of a single open reading frame that produces a single polyprotein that is cleaved, at multiple locations, by virus encoded and host cell proteases to yield about a dozen structural and nonstructural proteins. The structural proteins (vp1, vp3, vp0) expressed as a single P1 peptide are cleaved into vp0, vp3 and vp1, and self assemble into a non-enveloped icosahedral capsid that incorporates the viral genome.

In some aspects, the picornavirus virus may be a foot-and-mouth disease virus (FMDV). The production of FMDV structural proteins in cells has been difficult due to low expression levels only detectable by western blots. (Oem et al., “Characterization of recombinant foot-and-mouth disease virus pentamer-like structures expressed by baculovirus and their use as diagnostic antigens in a blocking ELISA,” Vaccine. 2007 May 16; 25(20):4112-21; Porta et al., “Efficient production of foot-and-mouth disease virus empty capsids in insect cells following down regulation of 3C protease activity,” J Virol Methods. 2013 February; 187(2):406-12.).

By adding a signal peptide sequence from A/Indonesia/5/05 HA protein to the N-terminus of the FMDV P1 structural protein precursor gene the P1 polypeptide expression levels in the baculovirus-insect cell expression system were significantly enhanced. Compare FIGS. 2A-2D, lanes 1-3, which lack the peptide, to lanes 4-6, which express the peptide. Signal peptides are found only on secretory and transmembrane proteins and because the FMDV proteins are neither secretory nor transmembrane proteins, the increased expression levels were particularly surprising. The signal peptide approach disclosed herein is thus particularly suitable for increasing expression of non-secretory proteins and non-transmembrane proteins.

In some aspects, portions of the protein may be removed during purification of the protein. In some aspects, cleavage is by a protease. The protease may be expressed within the host cell and may be an endogenous protease or an over-expressed protease. In other aspects, the fusion protein is cleaved after isolation from the host cell.

In particular aspects, the HA signal peptide may be removed. In yet other aspects, fusion proteins that are expressed as polyproteins may be cleaved into two or more individual proteins before administering to a subject. As an illustrative example, with respect to the FMDV polyprotein P1, the protein administered may be SEQ ID NO:4 with the hexahistidine tag removed and with the HA signal peptide (SEQ ID NO:3) removed. In other aspects, the polyprotein P1 is cleaved into the individual proteins vp1, vp2, vp3, and vp4 before administering to the subject.

In some aspects, purification of the disclosed fusion proteins may be facilitated by epitope tags. Suitable tags known in the art include a FLAG-tag, a six histidine tag (also known as hexahistidine or 6His), a Glu-Glu tag, a Glutathione-S-Transferase (GST) tag, and a maltose binding protein (MBP) tag. Any sequences disclosed herein that include epitope tags are also to be considered disclosed without the tag. The epitope tags may be located wherever required for purification. For example, the epitope tag may be on the N-terminus or on the C-terminus. In some aspects, the epitope tag may be contained within a fusion protein. Such a situation may occur, for example, when portions of the N- or C-terminus are removed during production of the fusion protein. In certain aspects, the epitope tag may be removed during or after protein synthesis.

In some aspects, fusion proteins may have a trans-membrane C-terminal (TM/CT) domain appended to the C-terminus of the immunogenic polypeptide. Suitable C-terminal domains are described in U.S. Patent Application Publication No. 2010-0184192, published Jul. 22, 2010. In some aspects, TM/CT domains may be derived from influenza HA. For example, suitable TM/CT domains may be derived from avian, pandemic and/or seasonal influenza virus. The TM/CT domain may derived from HA proteins in the group consisting of H1, H2, H3, H4, H5, H6, H7, H8, H9, H10, H11, H12, H13, H14, H15 and H16.

The immunogens (e.g., fusion proteins) may be administered in a variety of formulations. For example, the fusion proteins may be administered in a micelle, as a purified protein, or a virus-like particle (VLP). Micellar, pharmaceutical and vaccine formulations are also within the scope of various aspects of the disclosure.

### Cloning and Manipulation of Nucleic Acids

Aspects of the present disclosure are directed to nucleic acids that encode a protein. Methods of preparing nucleic acids encoding the proteins are known in the art. For example, a gene encoding a specific protein can be isolated by RT-PCR from polyadenylated mRNA extracted from cells. In some aspects, the resulting product gene can be cloned as a nucleic acid insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes. In some aspects, the vector may replicate autonomously. A vector may also be integrated into a chromosome of a host cell. In other aspects, the vector may be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, which does not replicate autonomously. In many, but not all, common embodiments, the vectors of the present disclosure are plasmids or bacmids.

General texts that describe molecular biological techniques, which are applicable to the present disclosure, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”).

In some aspects, nucleotides that encode proteins, including chimeric molecules, may be cloned into an expression vector. An “expression vector” is a vector, often a plasmid, which is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In another embodiment, the vector may further comprise nucleotides that encode additional proteins.

Suitable promoters for vectors include the AcMNPV polyhedrin promoter (or other baculovirus), phage lambda PL promoter, the E. coli lac, phoA and tac promoters, the SV40 early and late promoters, and promoters of retroviral LTRs. Other suitable promoters are known in the art. A vector may further contain sites for transcription initiation, termination, and, in the transcribed region, a ribosome-binding site for translation. The coding portion of the transcripts expressed by the constructs may include a translation initiating codon at the beginning and a termination codon appropriately positioned at the end of the polypeptide to be translated.

Vectors may include one or more selectable markers. Such markers include dihydrofolate reductase, G418 or neomycin resistance for eukaryotic cell culture and tetracycline, kanamycin or ampicillin resistance genes for culture in E. coli and other bacteria. Examples of vectors include virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used include bacterial vectors such as pQE70, pQE60 and pQE-9, pBluescript vectors, Phagescript vectors, pNH8A, pNH16a, pNH18A, pNH46A, ptrc99a, pKK223-3, pKK233-3, pDR540, and pRIT5. Among preferred eukaryotic vectors are pFastBacl pWINEO, pSV2CAT, p0044, pXT1, pSG, pSVK3, pBPV, pMSG, and pSVL. Other suitable vectors will be readily apparent to the skilled artisan.

Various types of mutagenesis can be used to alter amino acid or nucleotide sequences. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional methods include point mismatch repair, mutagenesis using repair-deficient host strains, restriction-selection and restriction-purification, deletion mutagenesis, mutagenesis by total gene synthesis, double-strand break repair, and the like. In one embodiment, mutagenesis can be guided by known information of the naturally occurring molecule or altered or mutated naturally occurring molecule, e.g., sequence, sequence comparisons, physical properties, crystal structure or the like.

In some aspects, the nucleotide sequences may contain silent mutations; e.g., to optimize codon expression for a particular host (change codons in the human mRNA to those preferred by insect cells such as Sf9 cells). See, for example, U.S. patent publication 2005/0118191.

Eukaryotic host cells include yeast, insect, avian, plant, C. elegans (or nematode) and mammalian host cells. Suitable examples of insect cells are Spodoptera frugiperda (Sf) cells, e.g. SD, Sf21, Trichoplusia ni cells, e.g. High Five cells, and Drosophila S2 cells. Examples of fungi (including yeast) host cells are S. cerevisiae, Kluyveromyces lactis lactis, species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Examples of prokaryotic host cells include bacterial cells, for example, E. coli, B. subtilis, Salmonella typhi, and mycobacteria.

### Virus-Like Particles

A variety of VLPs are known in the art. In some aspects, the fusion proteins of the disclosure are incorporated into VLPs. In other aspects, VLP production may be enhanced by using fusion proteins of the disclosure to enhance expression of proteins that assemble into VLPs. In some aspects, the VLP may be an influenza VLP. Influenza VLPs may be prepared as described in U.S. Pat. No. 7,763,450 to Robinson, U.S. Pat. No. 8,080,255 to Smith, U.S. Pat. No. 7,556,940 to Galarza, and Patent Application Publication No 2010/0129401. Expression of influenza M1 alone is sufficient to assemble VLPs. Accordingly, in addition to the one or more fusion proteins disclosed herein, an influenza VLP may include only the influenza M1 protein. In other aspects, one or more additional influenza proteins may be included. For example, additional proteins may include other influenza proteins such as M2, HA or NA.

The HA and NA proteins may be derived from different sub-types and/or different strains. For example, the HA may be selected from the group consisting of H1, H2, H3, H4, H5, H6, H7, H8, H9, H10, H11, H12, H13, H14, H15 and H16. The NA may be selected from the group consisting of N1, N2, N3, N4, N5, N6, N7, N8 and N9. The HA may exhibit hemagglutinin activity. The NA may exhibit neuraminidase activity.

The influenza proteins (e.g., M1, M2, HA, or NA) may be derived from various sources. For example, the influenza protein may be derived from an influenza infecting humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild, and game birds such as chickens, turkeys, ducks, and geese. In particular aspects, the influenza is an avian influenza; for example, a H9N2 sub-type (e.g., strain A/Hong Kong/1073/99) or a H5N1 sub-type (e.g., strain A/Indonesia/5/05). In other particular aspects, the influenza is an H1N1 sub-type or a H3N2 sub-type.

### Pharmaceutical or Vaccine Formulations and Administration

The proteins produced by the methods disclosed herein may be formulated as pharmaceutical compositions for administering to a subject in accordance with known approaches in the art.

The pharmaceutical compositions may contain a pharmaceutically acceptable carrier, diluent or excipient. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic compositions for inducing a protective immune response in a vertebrate.

In some aspects, formulations may include a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation may be adapted to suit the mode of administration. In a exemplary embodiment, the formulation is suitable for administration to humans, is sterile, non-particulate and/or non-pyrogenic.

The composition may also contain wetting agents, or emulsifying agents, or pH buffering agents, or mixtures thereof. The composition can be a solid form, such as a lyophilized powder suitable for reconstitution (e.g., with water or saline), a liquid solution, suspension, emulsion, tablet, pill, capsule, sustained release formulation, or powder. Oral formulations may include standard carriers such as pharmaceutical grades of mannitol, lactose, starch, magnesium stearate, sodium saccharine, cellulose, magnesium carbonate, etc.

In some aspects, a pharmaceutical pack or kit comprising one or more containers filled with one or more of the components of the formulations is provided. In a particular aspect, the kit may include two containers, a first container containing a toxin receptor-binding protein fusion protein, a micelle comprising toxin receptor-binding protein fusion protein, or a VLP comprising a toxin receptor-binding protein fusion protein, and a second container containing an adjuvant. Associated with such container(s) may be a notice in the form prescribed by a governmental agency regulating the manufacture, use or sale of pharmaceuticals or biological products, which notice reflects approval by the agency of manufacture, use or sale for human administration. Formulations may also be packaged in a hermetically sealed container such as an ampoule or sachette indicating the quantity of composition.

### Administration and Dosing

Administration may be any suitable route. Suitable routes include parenteral administration (e.g., intradermal, intramuscular, intravenous and subcutaneous), epidural, and mucosal (e.g., intranasal and oral or pulmonary routes or by suppositories), transdermally or intradermally. Administration may be by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some aspects, intranasal or other mucosal routes of administration may result in an antibody or other immune response that is substantially higher than other routes of administration. Administration can be systemic or local.

In some aspects, administration may be by injection using a needle and syringe, by a needle-less injection device. In other aspects, administration is by drops, large particle aerosol (greater than about 10 microns), or by spray into the upper respiratory tract.

In some aspects, administration may be targeted. For example, immunogen may be administered in such a manner as to target mucosal tissues in order to elicit an immune response at the site of immunization. Mucosal tissues such as gut associated lymphoid tissue (GALT) can be targeted for immunization by using oral administration of compositions which contain adjuvants with particular mucosal targeting properties. Additional mucosal tissues can also be targeted, such as nasopharyngeal lymphoid tissue (NALT) and bronchial-associated lymphoid tissue (BALT).

In some aspects, multiple immunogens may be administered. Where more than one immunogen is administered, the immunogens may be co-administered simultaneously to the same position of the subject; for example, by injection of material from a container containing multiple immunogens. In other aspects, when more than one immunogen is co-administered, they may be co-administered sequentially at different sites within a short space of time; for example, one administration may be in the thigh, and a second administration may be in the arm, with both administrations occurring within a short period (e.g. up to 30 minutes).

Administration may occur on a dosage schedule, for example, an initial administration of a composition with subsequent booster administrations. In particular embodiments, a second dose of the composition is administered from two weeks to one year, preferably from about 1, about 2, about 3, about 4, about 5, or about 6 months, after the initial administration. Additionally, a third dose may be administered after the second dose and from about three months to about two years, or even longer, preferably about 4, about 5, or about 6 months, or about 7 months, or about one year after the initial administration. The third dose may be optionally administered when no or low levels of specific immunoglobulins are detected in the serum and/or urine or mucosal secretions of the subject after the second dose. In a preferred embodiment, a second dose is administered about one month after the first administration and a third dose is administered about six months after the first administration. In another embodiment, the second dose is administered about six months after the first administration. In another embodiment, the compositions of the disclosure can be administered as part of a combination therapy. For example, compositions may be formulated with other immunogenic compositions, antivirals and/or antibiotics.

Pharmaceutical composition dosage may be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. The dosages can be determined from animal studies. A non-limiting list of animals used to study the efficacy of vaccines include the guinea pig, hamster, ferrets, chinchilla, mouse and cotton rat.

Most animals are not natural hosts to infectious agents but can still serve in studies of various aspects of the disease. For example, any of the above animals can be dosed with a vaccine candidate to partially characterize the immune response induced, and/or to determine if any neutralizing antibodies have been produced. For example, many studies have been conducted in the mouse model because mice are small size and their low cost allows researchers to conduct studies on a larger scale.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems. Dose may be adjusted based on, e.g., age, physical condition, body weight, sex, diet, time of administration, and other clinical factors.

While stimulation of immunity with a single dose is possible, additional dosages may be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections. Similarly, adults who are particularly susceptible to repeated or serious infections, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

In some aspects, the composition may be supplied as a liquid. The liquid form of the composition may be supplied in a hermetically sealed container at least about 50 μg/ml, more preferably at least about 100 μg/ml, at least about 200 μg/ml, at least 500 μg/ml, or at least 1 mg/ml.

### Adjuvants

The immunogenicity of a particular composition may be enhanced by the use of non-specific stimulators of the immune response, known as adjuvants. Adjuvants have been used experimentally to promote a generalized increase in immunity against antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2nd Edition),” herein incorporated by reference in its entirety for all purposes, is envisioned within the scope of this disclosure.

Exemplary adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/Tween® 80 emulsion.

In some aspects, the adjuvant may be a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiment, the vesicles are Novasomes®. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928.

Compositions of the disclosure may also be formulated with “immune stimulators.” These are the body's own chemical messengers (cytokines) to increase the immune system's response. Immune stimulators include, but not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CM); and other immunostimulatory molecules, such as macrophage inflammatory factor, Flt3 ligand, B7.1; B7.2, etc. The immunostimulatory molecules may be administered in the same formulation as the compositions of the disclosure, or may be administered separately. Either the protein or an expression vector encoding the protein may be administered to produce an immunostimulatory effect. Thus, in one embodiment, the disclosure comprises antigenic and vaccine formulations comprising an adjuvant and/or an immune stimulator. The present disclosure contemplates vaccinating humans and other animals with bacterial toxins to provide substantial immunity against subsequent disease induced by toxin introduced to the human or other animal as a result of infection by a toxin-producing organism or as a consequence of an event that introduces a toxin into the body.

Aspects of the disclosure are further illustrated by the following examples that should not be construed as limiting. The contents of all references, Accession No. sequences, patents and published patent applications cited throughout this application, as well as the Figures and the Sequence Listings therein, are incorporated herein by reference for all purposes.

### Example 1

### Preparation and Expression of FMDV Polyprotein

The FMDV P1 polyprotein was cloned into two vectors, BV1168 and BV1169 (FIG. 1). The vectors are identical except that BV1169 contained the A/Indonesia/5/05 signal peptide sequence at the N-terminus (SEQ ID NO:3; MEKIVLLLAIVSLVK). In both cases, the P1 polyprotein contained an N-terminal His6 tag. The protein and nucleotide sequence encoded by BV1168 are shown in FIGS. 4C and 4D, respectively. The protein and nucleotide sequence encoded by BV1168 are shown in FIGS. 4A and 4B, respectively.

Sf9 cells were infected with three clones of BV1168 and BV1169 at an MOI of 0.5 ffu/cell and incubated at 27° C., 150 rpm for ˜70 hours. Crude harvest (cells and media) were analyzed by SDS-PAGE and western blot as shown in FIG. 2. The lanes are as follows: M-Marker. Lanes 1-3 show FMDV P1 protein with a hexa-histidine tag with out the signal peptide expressed from BV1168. Lanes 4-6 show FMDV P1 protein with a hexa-histidine tag and the signal peptide expressed from BV1169. Lane 7 shows a BV266 control and lane 8 shows BV 1064 S300 Fxn. FIG. 2A shows the enhanced expression in lanes 4-6 by total protein stain in SDS-PAGE. FIGS. 2B-D shows enhanced expression in lanes 4-6 detected by anti-Histidine antibody (FIG. 2B; “anti HIS Mab”), anti-FMDV vp2 antibody (FIG. 2C; “F1412SA Mab”), and by anti-FMDV vp1 antibody (FIG. 2D; “12FE9.2.1A Mab”).

The data demonstrates that FMDV polyprotein P1 expression was substantially elevated when over-expressed as a fusion protein containing the N-terminal signal peptide. The P1 protein is produced in uncleaved form and is detectable by anti-FMDV monoclonal antibodies.

### Example 2

### Enhanced Expression of Single FMDV Polypeptide

Expression of individual non-secretory proteins is enhanced when fused to a signal peptide. Sf9 cells were infected with Baculovirus expressing FMDV vp0 or FMDV vp1 proteins each with and without a signal peptide. Cells were harvested 70 hours post-infection. The harvested crude material (i.e. cells and medium) was analyzed by SDS-PAGE and by western blot using monoclonal antibody specific to the recombinant protein. FIG. 3, panel (a), the left panel, shows expression of FMDV vp0. FIG. 3, panel (b), the right panel, shows expression of FMDV vp1 protein. In each case, “+” recombinant protein contained a signal peptide, “−” recombinant protein did not have a signal peptide. The arrow indicates the expressed vp1 protein.

### Example 3

### Expression of FMDV Polypeptide Containing Single and Multiple proteins

The signal peptide increases expression of expression of single proteins and proteins in tandem. FIG. 5 shows elevated expression with a variety of different constructs. Sf9 cells were infected with recombinant baculovirus (BV) expressing proteins 1, 2, 3, and/or 4 with (+) or without (−) a signal peptide and harvested ˜65 hrs post-infection. Crude samples (i.e. cells and medium) were harvested and analyzed by SDS-PAGE and western blots. The expressed recombinant proteins are indicated by the dot. BV1 expresses FMDV vp1. BV2 expresses FMDV vp0 vp3. BV3 expresses FMDV vp1, vp0 and vp3. BV4 expresses the FMDV P1 polyprotein (i.e., vp0-vp3-vp1). FIG. 5A shows a total protein stain. FIGS. 5B and 5C show binding of vp1 and vp2 antibodies, respectively. Note that the vp0 protein contains the vp2 protein. In each case, elevated expression of the proteins was obtained.

1. A fusion protein comprising
(a) an N-terminal signal peptide,
wherein the N-terminal signal peptide consists of SEQ ID NO:3; and
(b) an immunogenic polypeptide.

2. The fusion protein of claim 1 wherein the immunogenic polypeptide comprises a protein selected from the group consisting of a viral protein, a bacterial protein, and a fungal protein.

3. The fusion protein of claim 2 wherein the immunogenic polypeptide comprises a viral protein.

4. The fusion protein of claim 1 wherein the immunogenic polypeptide is a polyprotein comprising two, three, four, five or six proteins.

5. The fusion protein of claim 3 wherein the virus is selected from the group consisting of: a picornavirus, a flavirus, a togavirus, and a human papilloma virus.

6. The fusion protein of claim 3 wherein the viral protein comprises a Foot and Mouth Disease Virus (FMDV) protein.

7. The fusion protein of claim 4 wherein the polyprotein comprises at least two proteins selected from the group of proteins consisting of: a FMDV vp1 protein, a FMDV vp2 protein, a FMDV vp3 protein, and a FMDV vp4 protein.

8. The fusion protein of claim 1 further comprising an epitope tag.

9. The fusion protein of claim 8 wherein the epitope tag is selected from the group consisting of: a hexahistidine tag, a FLAG tag, a Glu-Glu tag, a Glutathione-S-Transferase (GST) tag, and a maltose binding protein (MBP) tag.

10. The fusion protein of claim 6 wherein the epitope tag is located on the N-terminus of the signal peptide.

11. The fusion protein of claim 6 wherein the epitope tag is located on the C-terminus of the immunogenic peptide.

12. The fusion protein of claim 1 wherein the immunogenic peptide is an FMDV protease.

13. A polynucleotide encoding the fusion protein of claim 1.

14. A host cell comprising the polynucleotide of claim 13.

15. An immunogenic composition comprising
(i) the fusion protein of claim 1 and
(ii) an adjuvant.

16. The immunogenic composition of claim 15 wherein the adjuvant is selected from the group consisting of: paucilamellar nonphospholipid vesicles, aluminum hydroxide, GMCSP, BCG, thur-MDP, nor-MDP, MTP-PE, monophosphoryl lipid A (MPL), MF-59, and RIM.

17. The composition of claim 15 further comprising a pharmaceutically acceptable carrier or diluent.

18. A method of increasing production of an immunogenic polypeptide comprising expressing the immunogenic polypeptide in a host cell as a fusion protein, wherein the fusion protein comprises
(a) an N-terminal signal peptide,
wherein the N-terminal signal peptide consists of SEQ ID NO:3; and
(b) the immunogenic polypeptide,
wherein production is increased compared to expressing the immunogenic polypeptide in the host cell without the N-terminal signal peptide.

19. The method of claim 18 wherein the host cell is a baculovirus cell.

20. A method of inducing a protective immune response in a subject comprising administering the composition of claim 15 to the subject.

21. The method of claim 20 wherein the subject is a human.

22. The method of claim 20 wherein the protective immune response comprises at least one of a cell-mediated immune response and an antibody-mediated immune response.

23. The method of claim 22 wherein the antibody-mediated immune response comprises neutralizing antibodies.


SEQUENCE LISTING (6 sequences)

SEQ ID NO: 1 - DNA, 2316 bp, Artificial Sequence
  BV1169 plasmid encoding FMDV protein P1 (VP1-4), HA signal peptide, and His6 tag
atggaaaagatcgtgctcctcctcgctatcgtgtccctggtcaagatgggtgccggtcaatcgtcgccagctacaggctcgcaaaaccagtcgggtaacacaggctctatcatcaacaactactacatgcaacagtaccaaaacagcatggacacccagctgggagataacgctatctctggtggcagcaacgagggttcgacagataccacttccacccacacaaccaacactcaaaacaacgactggttctccaagttggcctcctctgctttctctggcctgttcggagccctgctcgctgataagaagaccgaggaaactacattgctggaggaccgtatcctgaccactagaaacggccacacaacctccactacacagagctcagttggcgtgacatacggatacgccaccgctgaagacttcgtgtctggacccaacactagcggtttggagacacgtgtggtccaagctgaacgcttcttcaagacacacctgttcgactgggtgacctcggattccttcggtagatgccacctcttggagctccctaccgaccacaagggtgtctacggctccttgactgattcttacgcctacatgaggaacggttgggacgtcgaagttaccgctgtgggcaaccagttcaacggaggttgcctgctcgtggccatggtccccgagctctgttctatccagaagagggaattgtaccaactgaccctcttcccccaccagttcatcaaccctagaactaacatgacagctcacatcactgtgccattcgtgggcgtcaaccgttacgaccaatacaaggtccacaagccctggaccctggttgtgatggtcgttgcccccctcaccgtgaacactgagggtgctcctcagatcaaggtctacgccaacatcgctcccactaacgttcacgtggccggagagttcccaagcaaggaaggtatcttcccagtggcttgctcagacggatacggaggactggtcaccactgacccaaagaccgctgatccggtttacggcaaggtgttcaaccccccccgtaaccagctccctggcagattcactaacttgctggacgtggctgaggcttgtccaacattcctgcgcttcgaaggtggcgttccgtacgtgacaaccaagaccgactcagatagggtcttggctcagttcgacatgtcactggctgccaagcacatgtcgaacactttcctcgccggattggctcagtactacacccaatactccggtactatcaacccccacttcatgttcacaggccctaccgacgccaaggctcgctacatgatcgcttacgctccaccgggaatggagccacctaagaccccagaagctgccgctcactgcatccacgccgagtgggacactggtctgaactcaaagttcacattctcgatcccctacctctccgccgctgactacgcctacaccgcttctgatgtggctgaaactacaaacgtccagggttgggtttgtctgttccaaatcactcacggcaaggctgacggcgatgctctggtggtcctcgctagcgctggcaaggacttcgagttgcgtctgcctgtggatgcccgcgctgagaccactagcgctggcgaatcagctgacccagtgacaaccactgtcgaggattacggaggtgaaactcagatccaacgtcgccagcacacagacgtgtctttcatcatggatcgtttcgtcaaggttaccccacagaaccaaatcaacatcctcgacttgatgcaaatcccgagccacacactggtcggagctctcttgagagcttctacctactacttcagcgatttggagatcgccgtcaagcacgaaggagacctgacctgggttccaaacggcgctccggagaagggattggacaacacaaccaacccaactgcctaccacaaggctccgctgacccgtctggctctcccctacactgctcctcaccgcgttctcgctaccgtgtacaacggagaatgccgttactcacgcaacgccgtccccaacgttcgtggtgacttgcaggtgctggctcaaaaggttgtgcgcactctgcctacatcgttcaactacggtgccatcaaggctacccgcgtcactgagctgctctacaggatgaagagagccgaaacttactgccccaggcctttgctggccatccaccccacagaggctagacacaagcagaagatcgtggctcctgtcaagcaaaccctcaacttcgacctcttgaagttggccggcgatgtgacagagcaccaccaccaccaccactaa

SEQ ID NO: 2 - protein, 771 aa, Artificial Sequence
  FMDV protein P1 (VP1-4) HA signal peptide and His6 tag
MEKIVLLLAIVSLVKMGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTYGYATAEDFVSGPNTSGLETRVVQAERFFKTHLFDWVTSDSFGRCHLLELPTDHKGVYGSLTDSYAYMRNGWDVEVTAVGNQFNGGCLLVAMVPELCSIQKRELYQLTLFPHQFINPRTNMTAHITVPFVGVNRYDQYKVHKPWTLVVMVVAPLTVNTEGAPQIKVYANIAPTNVHVAGEFPSKEGIFPVACSDGYGGLVTTDPKTADPVYGKVFNPPRNQLPGRFTNLLDVAEACPTFLRFEGGVPYVTTKTDSDRVLAQFDMSLAAKHMSNTFLAGLAQYYTQYSGTINPHFMFTGPTDAKARYMIAYAPPGMEPPKTPEAAAHCIHAEWDTGLNSKFTFSIPYLSAADYAYTASDVAETTNVQGWVCLFQITHGKADGDALVVLASAGKDFELRLPVDARAETTSAGESADPVTTTVEDYGGETQIQRRQHTDVSFIMDRFVKVTPQNQINILDLMQIPSHTLVGALLRASTYYFSDLEIAVKHEGDLTWVPNGAPEKGLDNTTNPTAYHKAPLTRLALPYTAPHRVLATVYNGECRYSRNAVPNVRGDLQVLAQKVVRTLPTSFNYGAIKATRVTELLYRMKRAETYCPRPLLAIHPTEARHKQKIVAPVKQTLNFDLLKLAGDVTEHHHHHH

SEQ ID NO: 3 - protein, 15 aa, Artificial Sequence
  HA signal peptide from A/Indonesia/5/05
MEKIVLLLAIVSLVK

SEQ ID NO: 4 - DNA, 2271 bp, Artificial Sequence
  BV1168 plasmid encoding FMDV protein P1 (VP1-4) and His6 tag
atgggtgccggtcaatcgtcgccagctacaggctcgcaaaaccagtcgggtaacacaggctctatcatcaacaactactacatgcaacagtaccaaaacagcatggacacccagctgggagataacgctatctctggtggcagcaacgagggttcgacagataccacttccacccacacaaccaacactcaaaacaacgactggttctccaagttggcctcctctgctttctctggcctgttcggagccctgctcgctgataagaagaccgaggaaactacattgctggaggaccgtatcctgaccactagaaacggccacacaacctccactacacagagctcagttggcgtgacatacggatacgccaccgctgaagacttcgtgtctggacccaacactagcggtttggagacacgtgtggtccaagctgaacgcttcttcaagacacacctgttcgactgggtgacctcggattccttcggtagatgccacctcttggagctccctaccgaccacaagggtgtctacggctccttgactgattcttacgcctacatgaggaacggttgggacgtcgaagttaccgctgtgggcaaccagttcaacggaggttgcctgctcgtggccatggtccccgagctctgttctatccagaagagggaattgtaccaactgaccctcttcccccaccagttcatcaaccctagaactaacatgacagctcacatcactgtgccattcgtgggcgtcaaccgttacgaccaatacaaggtccacaagccctggaccctggttgtgatggtcgttgcccccctcaccgtgaacactgagggtgctcctcagatcaaggtctacgccaacatcgctcccactaacgttcacgtggccggagagttcccaagcaaggaaggtatcttcccagtggcttgctcagacggatacggaggactggtcaccactgacccaaagaccgctgatccggtttacggcaaggtgttcaaccccccccgtaaccagctccctggcagattcactaacttgctggacgtggctgaggcttgtccaacattcctgcgcttcgaaggtggcgttccgtacgtgacaaccaagaccgactcagatagggtcttggctcagttcgacatgtcactggctgccaagcacatgtcgaacactttcctcgccggattggctcagtactacacccaatactccggtactatcaacccccacttcatgttcacaggccctaccgacgccaaggctcgctacatgatcgcttacgctccaccgggaatggagccacctaagaccccagaagctgccgctcactgcatccacgccgagtgggacactggtctgaactcaaagttcacattctcgatcccctacctctccgccgctgactacgcctacaccgcttctgatgtggctgaaactacaaacgtccagggttgggtttgtctgttccaaatcactcacggcaaggctgacggcgatgctctggtggtcctcgctagcgctggcaaggacttcgagttgcgtctgcctgtggatgcccgcgctgagaccactagcgctggcgaatcagctgacccagtgacaaccactgtcgaggattacggaggtgaaactcagatccaacgtcgccagcacacagacgtgtctttcatcatggatcgtttcgtcaaggttaccccacagaaccaaatcaacatcctcgacttgatgcaaatcccgagccacacactggtcggagctctcttgagagcttctacctactacttcagcgatttggagatcgccgtcaagcacgaaggagacctgacctgggttccaaacggcgctccggagaagggattggacaacacaaccaacccaactgcctaccacaaggctccgctgacccgtctggctctcccctacactgctcctcaccgcgttctcgctaccgtgtacaacggagaatgccgttactcacgcaacgccgtccccaacgttcgtggtgacttgcaggtgctggctcaaaaggttgtgcgcactctgcctacatcgttcaactacggtgccatcaaggctacccgcgtcactgagctgctctacaggatgaagagagccgaaacttactgccccaggcctttgctggccatccaccccacagaggctagacacaagcagaagatcgtggctcctgtcaagcaaaccctcaacttcgacctcttgaagttggccggcgatgtgacagagcaccaccaccaccaccactaa

SEQ ID NO: 5 - protein, 756 aa, Artificial Sequence
  FMDV protein P1 (VP1-4) and His6 tag
MGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTYGYATAEDFVSGPNTSGLETRVVQAERFFKTHLFDWVTSDSFGRCHLLELPTDHKGVYGSLTDSYAYMRNGWDVEVTAVGNQFNGGCLLVAMVPELCSIQKRELYQLTLFPHQFINPRTNMTAHITVPFVGVNRYDQYKVHKPWTLVVMVVAPLTVNTEGAPQIKVYANIAPTNVHVAGEFPSKEGIFPVACSDGYGGLVTTDPKTADPVYGKVFNPPRNQLPGRFTNLLDVAEACPTFLRFEGGVPYVTTKTDSDRVLAQFDMSLAAKHMSNTFLAGLAQYYTQYSGTINPHFMFTGPTDAKARYMIAYAPPGMEPPKTPEAAAHCIHAEWDTGLNSKFTFSIPYLSAADYAYTASDVAETTNVQGWVCLFQITHGKADGDALVVLASAGKDFELRLPVDARAETTSAGESADPVTTTVEDYGGETQIQRRQHTDVSFIMDRFVKVTPQNQINILDLMQIPSHTLVGALLRASTYYFSDLEIAVKHEGDLTWVPNGAPEKGLDNTTNPTAYHKAPLTRLALPYTAPHRVLATVYNGECRYSRNAVPNVRGDLQVLAQKVVRTLPTSFNYGAIKATRVTELLYRMKRAETYCPRPLLAIHPTEARHKQKIVAPVKQTLNFDLLKLAGDVTEHHHHHH

SEQ ID NO: 6 - protein, 500 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRN